(19) United States
(12) Patent Application Publication
(10) Pub. No.: US 2009/0226374 A1
(43) Pub. Date: Sep. 10, 2009

(54) ANAPHYLATOXINS FOR DETECTING CLINICAL CONDITIONS

(75) Inventor: Tony E. Hugli, San Diego, CA (US)

Correspondence Address: DLA PIPER LLP (US), 4365 EXECUTIVE DRIVE, SUITE 1100, SAN DIEGO, CA, 92121-2133, US

(73) Assignee: Health Aide, Inc., San Diego, CA (US)

(21) Appl. No.: 12/356,423

(22) Filed: Jan. 20, 2009

Related U.S. Application Data
(63) Continuation-in-part of application No. 10/975,323, filed on Oct. 27, 2004.
(60) Provisional application No. 60/514,716, filed on Oct. 27, 2003.

(51) Int. Cl. A61K 49/00; C12Q 1/02; C07K 7/08; C07K 7/06; C07K 5/10; C07K 5/08

(52) U.S. Cl. 424/9.2; 435/29; 530/328; 530/327; 530/326; 530/329; 530/330; 530/331

(57) ABSTRACT

Non-allergic hypersensitivity reactions can be observed in a sample of cells from a subject in response to anaphylatoxins. Accordingly, methods are provided for detecting clinical conditions such as cellular hyper-reactivity, non-allergic hypersensitivity, asthma, inflammation, chronic or acute infection, bacterial infection, viral infection, parasite infection, adverse drug reaction, organ rejection, vasculitis, mastocytosis, eosinophilia, basophilia, leukemia, and/or C3a or C5a receptor defects in a subject. Also provided are kits for detecting such clinical conditions in a subject.

### CROSS REFERENCE TO RELATED APPLICATION(S)

This application is a continuation-in-part of U.S. Ser. No. 10/975,323, filed Oct. 27, 2004, now pending which claims the benefit of priority under 35 U.S.C. § 119(e) of U.S. Ser. No. 60/514,716, filed Oct. 27, 2003. The disclosure of each of the prior applications is considered part of and is incorporated by reference in the disclosure of this application.

### BACKGROUND OF THE INVENTION

1. Field of the Invention

The present invention relates generally to medical diagnostics and more specifically to methods and compositions using blood or body fluids for identifying an individual that have a clinical condition and/or may be hypersensitive or hyper-reactive to a foreign compound, therapeutic compound, or clinical/therapeutic procedures.

2. Background Information

The incidence of allergy in the human population is universally high. Unfortunately, the incidence of pseudo-allergic reactions (i.e., idiopathic or atypical hypersensitivity reactions) in the human population is also high and many of these reactions are classified as “complement-related” pseudo-allergy responses. Recent estimates of the frequency of pseudo-allergy predicts that as many as 420,000 severe pseudo-allergic reactions every year in the United States with 20,400 fatalities.

During complement activation, the 74-77 amino acid fragments C3a, C4a and C5a are released. They are potent inflammatory mediators, inducing cellular degranulation, smooth muscle contraction, arachidonic acid metabolism, cytokine release, and cellular chemotaxis, and have been implicated in the pathogenesis of a number of inflammatory diseases. These peptides are designated “anaphylatoxins” for historical reasons and because of their ability to elicit a systemic reaction in guinea pigs that closely resembles acute anaphylactic shock. These fragments also cause the cellular release of histamine, vasoactive amines, and lysosomal enzymes. These biological activities implicate the anaphylatoxins as mediators in the inflammatory process and of tissue injury.

The diagnosis of allergies and pseudo-allergies to drugs and/or food represent one of the most frustrating problems for the allergist. Skin tests often do not work with drug allergens, IgE tests are often still missing, and cellular proliferation tests are complicated and very time consuming. Therefore, tests for the detection of drug allergies are rarely offered in the routine laboratory, in spite of contributing approximately 10% of cases that are examined by allergists, and about 3-5% of the population being affected by such allergies.

The standard allergy skin test is commonly used to predict potential allergic reactions. This skin test, often referred to as a “wheal and flare test”, includes administering a small amount of antigen in a localized area, and viewing the area for a localized reaction. More specifically, a small volume of antigen solution is injected into the skin and after twenty-four hours the injection site is visually examined for the presence of a raised circular bump, the wheal, and redness, the flare. The presence of a wheal or flare identifies the individual as having an allergy to the antigen. However, there are no reported skin tests or other forms of tests that can diagnose a clinical condition in a subject and/or identify individuals prone to non-atopic or non-immune hypersensitivity reactions (i.e., pseudo-allergic reactions). A need therefore exists for methods and compounds for detecting such clinical conditions in a subject.

### SUMMARY OF THE INVENTION

The present invention is based, in part, on the observation that cellular reactions, such as non-allergic hypersensitivity reactions, can be observed in the cells of a subject in response to contact with anaphylatoxins. Thus, the present invention addresses the need for a test to identify individuals having a clinical condition and/or individuals prone to non-immune hypersensitive reactions (i.e., pseudo-allergic reactions). The test includes contacting a sample of cells from the subject with an anaphylatoxin or analogue thereof, and detecting a cellular reaction, wherein the reaction is indicative of a clinical condition, such as non-allergic hypersensitivity in the subject. In one embodiment, the method is performed ex vivo. In another embodiment, the cells are tissue or blood cells, such as whole blood or isolated blood cells. In another embodiment, the reaction occurs within 1-24 hours of contacting the cells with the stimulant or activator. In another embodiment, the reaction is expression of one or more cell factors such as, but not limited to, cytokines (e.g., interleukins, eotaxins, and chemokines), metabolic factors (e.g., vasoamines and arachidonate products), and cell markers including growth factors.

The results from performing the methods of the invention on one or more subjects can be compared to results obtained from cells that are from the same organ and/or of the same cell type as the subject being tested as a way to determine whether the subject being tested is a high responder. In another embodiment, the results can be compared to results from corresponding normal cells or cells from a subject that is known to be a low responder in order to identify the subject being tested as a high responder. Thus, the magnitude of the difference in cellular response between a test subject and that of corresponding normal cells or cells from a subject that is known to be a low responder may be indicative of one or more clinical conditions.

In one embodiment, the present invention allows for the detection of non-allergic hypersensivity to a variety of potential compounds, drugs, medications or treatments such as, but not limited to, foreign infused radio-contrast media, infused immunoglobulin therapy, infused protein replacement therapy (including but not limited to serum albumin and Factor VIII), infused recombinant plasma proteins (such as but not limited to serum albumin, infused plasma coagulation proteins, proteinase inhibitors), or general blood substitutes as known in the biological and chemical arts. The non-allergic reaction may be indicative of systemic hypersensitivity or hyper-responsiveness (i.e., pseudo-allergy). Thus, the reaction can be indicative of adverse reactivity towards oral or intravenous drug treatments; adverse reactivity towards infused radio-contrast media or infused non-protein treatments; or non-IgE anaphylatoid reactions, abnormalities of mast cells, basophils, eosinophils, monocytes or neutrophils, abnormal reactivity towards inflammatory mediators (including secondary inflammatory mediators released from cells in response to the anaphylatoxins), and abnormal reactivity towards infused recombinant proteins such as immunoglobulins and/or antibodies. In another embodiment, the present invention allows for the detection of a clinical condition such as asthma, inflammation, chronic or acute infection, bacterial infection, viral infection, parasite infection, adverse drug reaction, organ rejection, vasculitis, mastocytosis, eosinophilia, basophilia, leukemia, and C3a or C5a receptor defects.

Examples of anaphylatoxins suitable for use in the present invention include, but are not limited to, C3a, C4a, or C5a, or an anaphylatoxin analogue or derivative thereof. Exemplary anaphylatoxin analogues include, but are not limited to peptides as set forth in SEQ ID NO: 1-92, analogue peptides of C5a, and organic small molecules that exhibit C3a or C5a activity.

The present invention further relates to a kit for detecting non-allergic hypersensitivity in a subject comprising at least one anaphylatoxin, such as C3a, C5a or analogues thereof. Exemplary analogues include peptides as set forth in SEQ ID NO: 1-92. The kit may further include a means for obtaining cells from the subject such as, for example, a needle and syringe, and may also include a means for contacting the cells with the anaphylatoxin.

The present invention further relates to a kit for detecting hyper-responsiveness in a subject comprising at least one anaphylatoxin, such as C3a, C5a or analogues thereof. Exemplary analogues include peptides as set forth in SEQ ID NO: 1-92. The kit may further include a means for obtaining cells from the subject such as, for example, a needle and syringe, and may also include a means for contacting the cells with the anaphylatoxin.

### DETAILED DESCRIPTION OF THE INVENTION

The present invention is based on the identification of compounds that trigger adverse reactions that do not involve the antibody- or immunoglobulin-mediated aspect of the immune system. Thus, the present invention discloses methods and compositions that identify individuals having a clinical condition and/or that are susceptible to non-atopic or non-immune hypersensitivity reactions. Further, the present invention is useful for identifying a population or a subpopulation of individuals that exhibit greater hypersensitivity to foreign compounds or treatments than the typical individual.

The present invention is not limited to the particular methodology, protocols, cell lines, vectors, reagents, and the like, described herein, as these may vary. It is also to be understood that the terminology used herein is used for the purpose of describing particular embodiments only, and is not intended to limit the scope of the present invention. As used herein and in the appended claims, the singular forms “a,”, “an,” and “the” include plural reference unless the context clearly dictates otherwise.

During allergic sensitization, Immunoglobulin E (IgE) antibodies are produced by white blood cells in response to initial exposure to an antigen (i.e, the causative allergen). IgE is found mainly in tissues, where, in complex with an antigen (i.e., on repeated exposure to the causative allergen), it activates the release of histamines from specialized, blood-derived cells called mast cells. Histamine release is the cause of such allergic reactions as hives, asthma, and hay fever. The tendency to develop allergic sensitization is referred to as “atopy”.

Non-atopic hypersensitivity reactions are those reproducible adverse reactions that do not involve the IgE immune system. As used herein, “idiopathic” refers to any disease or condition the cause of which is not known or that arises spontaneously. Accordingly, the present invention may be used to predict when severe idiopathic non-immune reactions may occur.

Identification of high responders may be performed in a clinic or medical office and may provide a physician with an individual's hypersensitive profile which may be useful in predicting the outcome of various treatments or assist in the identification of individuals “at risk” of hypersensitivity to a compound or treatment. An “at risk” patient may require special precautions during a treatment or procedure. As used herein, the term “high responder” refers to a subject that displays visible reactions to the agonist, and when subjected to a skin test, is identified by the presence or increased presence of a wheal or flare compared to the low responders. Alternatively, or in addition to use of a skin test, a high responder may be identified by detecting variable cellular responses to the activating factors C3a or C5a in a blood sample from the subject. High responders may then be identified as having a higher risk of hypersensitivity to a compound or treatment than a low responder. Thus, significant differences exist in the cellular responses to anaphylatoxins among individuals, and detecting these different physiologic/immunologic conditions of individuals and the respective magnitude of the differences could be useful both clinically and diagnostically.

The importance of identifying adverse side reactions of treatments is significant because in most treatments, the quantities of foreign materials or the amount of partially denatured or improperly folded protein materials are so high that activation of the host defense system (i.e., complement system) in high responders is inevitable. The mode of treatment administration is also of significance because the faster the materials are administered, the more activation occurs due to rapid decay of the meta-stable enzymes used as a biological means of auto-regulation and control. Thus, a seemingly harmless side reaction in a low responder could be lethal in a high responder. The physician can then utilize these results in determining appropriate therapies for such high responders.

As used herein, the term “agonist” refers to an agent or analog that is capable of inducing a full or partial pharmacological response. For example, an agonist may bind productively to a receptor and mimic the physiological reaction thereto. The term “antagonist” refers to an agent that is capable of inhibiting or otherwise reducing a pharmacological response. For example, an antagonist binds to receptors but does not provoke the normal biological response. Thus, an antagonist potentiates or recapitulates, for example, the bioactivity of a target gene, such as to repress transcription of the target genes.

As used herein, the term “antibody” is meant to include intact molecules of polyclonal or monoclonal antibodies, chimeric, single chain, and humanized antibodies, as well as fragments thereof, such as Fab and F(ab′)₂, Fv and SCA fragments that are capable of binding an epitopic determinant. Monoclonal antibodies are made from antigen containing fragments of the protein by methods well known to those skilled in the art (Kohler, et al., Nature, 256:495, 1975). An Fab fragment consists of a monovalent antigen-binding fragment of an antibody molecule, and can be produced by digestion of a whole antibody molecule with the enzyme papain, to yield a fragment consisting of an intact light chain and a portion of a heavy chain. An Fab′ fragment of an antibody molecule can be obtained by treating a whole antibody molecule with pepsin, followed by reduction, to yield a molecule consisting of an intact light chain and a portion of a heavy chain. Two Fab′ fragments are obtained per antibody molecule treated in this manner. An (Fab′)₂ fragment of an antibody can be obtained by treating a whole antibody molecule with the enzyme pepsin, without subsequent reduction. A (Fab′)₂ fragment is a dimer of two Fab′ fragments, held together by two disulfide bonds. An Fv fragment is defined as a genetically engineered fragment containing the variable region of a light chain and the variable region of a heavy chain expressed as two chains. A single chain antibody (“SCA”) is a genetically engineered single chain molecule containing the variable region of a light chain and the variable region of a heavy chain, linked by a suitable, flexible polypeptide linker.

The term “administration” or “administering” is defined to include an act of providing a compound or composition of the invention to the subject in need of treatment. The phrases “parenteral administration” and “administered parenterally” as used herein mean modes of administration other than enteral and topical administration, usually by injection, and include, without limitation, intravenous, intramuscular, intraarterial, intrathecal, intracapsular, intraorbital, intracardiac, intradermal, intraperitoneal, transtracheal, cutaneous and subcutaneous, subcuticular, intraarticulare, subcapsular, subarachnoid, intraspinal and intrasternal injection and infusion. The phrases “systemic administration,” “administered systemically,” “peripheral administration” and “administered peripherally” as used herein mean the administration of a compound, drug or other material other than directly into the central nervous system, such that it enters the subject's system and, thus, is subject to metabolism and other like processes, for example, inhalation or subcutaneous administration.

Accordingly, the invention provides methods of detecting a clinical condition such as non-allergic hypersensitivity in a subject. As used herein, “hypersensitivity” refers to the tendency to respond abnormally to the presence of a particular antigen, which may cause a variety of tissue reactions ranging from serum sickness to an allergy or, at the severest, to anaphylactic shock. As can be envisioned, the present invention may have a wide variety of applications. This test may detect all or some subjects prone to non-allergic hypersensitivity to foreign compounds or to pseudo-allergic reactions. As used herein, “pseudo-allergic” reactions refer to any reactions caused by complement activation products or other non-immune mechanisms leading to cellular activation events. As such, the methods of the invention can be used for detecting hyper-responsiveness in a subject. The term, “hyper-responsiveness” refers to the tendency of certain cells to respond abnormally to the presence of a particular mediator or activator and cause a pseudo-allergic reaction (i.e., a non-allergic reaction).

The term “subject” as used herein refers to any individual or patient to which the invention methods are performed. Generally the subject is human, although as will be appreciated by those in the art, the subject may be an animal. Thus other animals, including mammals such as rodents (including mice, rats, hamsters and guinea pigs), cats, dogs, rabbits, farm animals including cows, horses, goats, sheep, pigs, and primates (including monkeys, chimpanzees, orangutans and gorillas) are included within the definition of subject. However, the method can also be practiced in other species, such as avian species (e.g., chickens).

In one embodiment, the methods of the invention are performed prior to treatment with an infused material, a known drug, a novel drug, or a new drug. For example, the present invention may be utilized to predict hypersensitivity prior to receiving infused radio-contrast media, blood substitutes, immunoglobulins, serum albumin, and recombinant plasma proteins such as coagulation/hemostatic proteins, complement proteins, and proteinase inhibitor molecules. In another embodiment, the methods may be used prior to receiving infused cancer drugs to determine hypersensitivity of the subject to the drugs. Ingested materials, including a variety of drugs and medication, can also induce non-immune reactions that have been classified as pseudo-allergy responses or non-immune hypersensitivity reactions and may be applicable to the present skin test.

In another embodiment, the method of detecting a clinical condition in a subject includes an intradermal (e.g., cutaneous) injection of an anaphylatoxin that may cause a visible cutaneous response in the form of a raised circular bump on the skin surface (called a wheal) and a red area (called a flare) with symptoms of local pruritis. As used herein, “pruritis” refers to any itching caused by local irritation of the skin or sometimes nervous disorders.

Molecules useful in the methods of the invention include, but are not limited to anaphylatoxins, such as C3a, C4a, or C5a, and analogues thereof. The term “anaphylatoxin” as used herein refers to any activator or mediator that produces an abnormal reaction in which histamine is released from tissues and causes either local or widespread symptoms in a subject. A peptide analogue of an anaphylatoxin may be a natural or a synthetic peptide based on the structure of human C3a, C4a or C5a, an analogue C3a, C4a or C5a peptide, or a C3a, C4a or C5a analogue molecule that mimics the activity of the human C3a, C4a, or C5a anaphylatoxin molecule. Examples of potential peptides are provided in Tables 1A-1F. Other anaphylatoxin analogues include organic small molecules that are able to bind to and stimulate C3a and C5a receptors. Accordingly, use of the term “anaphylatoxins” includes anaphylatoxin molecules herein described and all natural and synthetic analogues and derivatives thereof.

The anaphylatoxins or analogues thereof should be of a molecular size that will not induce an immune response (i.e., be a hapten and non-antigenic). The anaphylatoxins or analogues thereof may mimic the actual structure of the natural factor or it may be a molecular design that can mimic the functional properties and actions of the natural agonist factors C3a, C4a or C5a (i.e., organic small molecules). The anaphylatoxins or analogues thereof may cause receptor-specific cellular activation, such as non-cytotoxic mast cell histamine release and/or the release of other inflammatory mediators and secondary mediators from skin mast cells and other cell types (e.g., blood cells). Amino acid residues within the structure (i.e., linear sequence) of the anaphylatoxin, analogue peptide or analogue molecule may be substituted using either: 1) natural amino acids, 2) non-natural amino acids, or 3) organic non-amino acid structures. The peptide must be salt free, endotoxin free and highly purified.

Examples of natural and analogue anaphylatoxin peptides of these three factors (i.e., C3a, C4a and C5a) have been described in the literature. Agonist peptides of C3a, C4a and C5a have each been shown to include the effector (i.e., receptor binding) site at the C-terminal portion of the molecule, such as Ala-Ser-His-Leu-Gly-Leu-Ala-Arg (SEQ ID NO: 1) which is the C-terminal octapeptide of human C3a (Hugli, T. E., Human Anaphylatoxin (C3a) from the Third Component of Complement, J. Biol. Chem., 250: pp. 8293-8301, 1975 and Hugli, T. E. and Erickson, B. W. Synthetic peptides with the biological activities and specificity of human C3a anaphylatoxin, Proc. Natl. Acad. Sci, USA., 74: pp. 1826-1830, 1977).

As used herein, the terms “analogue” and “analog” are used interchangeably to refer to any structural derivative of a parent compound that often differs from it by a single element, but retains the functionality of the parent. As used herein, the term “protein” refers to at least two covalently attached amino acids, which includes proteins, polypeptides, oligopeptides and peptides. A protein may be made up of naturally occurring amino acids and peptide bonds, or synthetic peptidomimetic structures. Thus, “amino acid”, or “peptide residue”, as used herein refers to both naturally occurring and synthetic amino acids. For example, homo-phenylalanine, citrulline and noreleucine are considered amino acids for the purposes of the invention. “Amino acid” also includes imino acid residues such as proline and hydroxyproline. The side chains may be in either the (R) or the (S) configuration.

The terms “polynucleotide”, “nucleic acid”, “nucleic acid sequence”, or “nucleic acid molecule” refer to a polymeric form of nucleotides at least four bases in length. The nucleotides of the invention can be deoxyribonucleotides, ribonucleotides in which uracil (U) is present in place of thymine (T), or modified forms of either nucleotide. The nucleotides of the invention can be complementary to the deoxynucleotides or to the ribonucleotides.

In another embodiment, the method of detecting a clinical condition such as non-allergic hypersensitivity in a subject includes an intradermal (e.g., cutaneous) injection of an anaphylatoxin, and further obtaining a sample of cells from the subject. Certain anaphylatoxins cause increased vascular permeability. Thus, the blood cells with C3a and/or C5a receptors can be monitored in biopsied skin cells. Any detectable skin reactions could be indicative of abnormalities in the circulating cell populations in a subject. As such, detection of responses of blood cells similar to the responses of the skin cells reflects and/or may be diagnostic of a number of known clinical conditions. Exemplary clinical conditions for which the present invention may be used for detection and/or monitoring include, but are not limited to, pseudo-allergy, asthma, allergy/allergic hypersensitivity/immuno-hypersensitivity, inflammatory conditions/general inflammation, chronic or acute infections, bacterial infections, viral infections, parasite infections, drug reactions, organ rejection, vasculitis, mastocytosis, eosinophilia, basophilia, leukemias, and C3a or C5a receptor defects/function.

The circulating basophils, eosinophils, monocytes and neutrophils all have C5a receptors and can migrate (e.g., chemotaxis) to the injection site and be counted in a tissue biopsy and/or can react with the cells of the subject to produce a detectable response. Similarly, basophils, eosinophils and monocyes have C3a receptors, which can be assessed for abnormalities. Any detectable cellular response could be indicative of abnormalities in the circulating cell populations in a subject, thereby detecting and/or monitoring one or more clinical conditions in the subject.

The anaphylatoxins of the invention may be administered to humans and other animals for detection of non-allergic hypersensitivity by any suitable route of administration, including orally, nasally, as by, for example, a spray, rectally, intravaginally, parenterally, intracistemally and topically, as by powders, ointments or drops, including buccally and sublingually. The peptides can be administered as such or in admixtures with pharmaceutically acceptable carriers, and can also be administered in conjunction with other peptides for detection of additive reactions.

Pharmaceutically acceptable carriers useful for formulating a peptide of the invention for administration to a subject are well known in the art and include, for example, aqueous solutions such as water or physiologically buffered saline or other solvents or vehicles such as glycols, glycerol, oils such as olive oil or injectable organic esters. A pharmaceutically acceptable carrier can contain physiologically acceptable compounds that act, for example, to stabilize or to increase the absorption of the peptide. Such pharmaceutically acceptable compounds include, for example, carbohydrates, such as glucose, sucrose or dextrans, antioxidants, such as ascorbic acid or glutathione, chelating agents, low molecular weight proteins or other stabilizers or excipients. One skilled in the art would know that the choice of a pharmaceutically acceptable carrier, including a physiologically acceptable compound, depends, for example, on the physico-chemical characteristics of the therapeutic agent and on the route of administration of the peptide, which can be, for example, orally or parenterally such as intravenously, and by injection, intubation, or other such method known in the art. The pharmaceutical composition also can contain a second (or more) compound(s) such as a diagnostic reagent, nutritional substance, toxin, or therapeutic agent, and/or vitamin(s).

In another embodiment, the method of detecting a clinical condition in a subject includes contacting cells from the subject with an anaphylatoxin and detecting a cellular response such as cytokine expression and/or one or more cell-derived factor profiles. The cells may be derived from a sample of tissue of bodily fluids from the subject being tested. In one embodiment, cytokine expression includes, but is not limited to, expression of interleukins, eotaxins, and chemokines. Exemplary cell-derived factors include, but are not limited to, metabolic factors (e.g., vasoamines and arachidonate products), and cell markers including growth factors. In another embodiment, the interleukin expressed is IL-5. In yet another embodiment, the vasoamine is histamine. Such responses reflect and/or are diagnostic of a number of known clinical conditions. As discussed above, exemplary clinical conditions for which the present invention may be used for detection and/or monitoring include, but are not limited to, pseudo-allergy, asthma, allergy/allergic hypersensitivity/immuno-hypersensitivity, inflammatory conditions/general inflammation, chronic or acute infections, bacterial infections, viral infections, parasite infections, drug reactions, organ rejection, vasculitis, mastocytosis, eosinophilia, basophilia, leukemias, and C3a or C5a receptor defects/function.

As used herein, the terms “sample” and “biological sample” refer to any sample suitable for the methods provided by the present invention. In one embodiment, the sample used in the methods of the present invention is a tissue sample, e.g., a biopsy specimen such as samples from needle biopsy (e.g., biopsy sample). In other embodiments, the sample used in the methods of the present invention is a sample of bodily fluid, e.g., blood, serum, plasma, sputum, lung aspirate, urine, or ejaculate.

In another embodiment, the anaphylatoxins of the invention may be contacted with cells of a subject ex vivo, for example, in a culture medium or on a solid support. When practiced as an in vitro assay, the methods can be adapted to a high throughput format, thus allowing the examination of a plurality (i.e., 2, 3, 4, or more) of cell samples and/or anaphylatoxins, which independently can be the same or different, in parallel. A high throughput format provides numerous advantages, including that anaphylatoxins can be tested on several samples of cells from a single patient, thus allowing, for example, for the identification of a particularly effective concentration of anaphylatoxin to be administered to the subject, or for the identification of a particularly effective anaphylatoxin for future monitoring of a particular clinical condition in the subject. As such, a high throughput format allows for the examination of two, three, four, etc., different anaphylatoxins, alone or in combination, on the cells (e.g., blood) of a subject such that the best (most effective) anaphylatoxin or combination of anaphylatoxins can be used for a further monitoring. Further, a high throughput format allows, for example, control samples (positive controls and or negative controls) to be run in parallel with test samples, including, for example, samples of cells known to be from high or low responders.

When performed in a high throughput (or ultra-high throughput) format, the method can be performed on a solid support (e.g., a microtiter plate, a silicon wafer, or a glass slide), wherein samples to be contacted with an anaphylatoxin are positioned such that each is delineated from each other (e.g., in wells). Any number of samples (e.g., 96, 1024, 10,000, 100,000, or more) can be examined in parallel using such a method, depending on the particular support used. Where samples are positioned in an array (i.e., a defined pattern), each sample in the array can be defined by its position (e.g., using an x-y axis), thus providing an “address” for each sample. An advantage of using an addressable array format is that the method can be automated, in whole or in part, such that cell samples, reagents, anaphylatoxins, and the like, can be dispensed to (or removed from) specified positions at desired times, and samples (or aliquots) can be monitored, for example, for detectable reactions.

Once a clinical condition or disease is established and a treatment protocol is initiated, subsequent assays may be performed on a regular basis to evaluate whether the cellular reaction (e.g., level of expression of the cytokines and/or cellular factors) in the subject begins to approximate that which is observed in the normal patient. The results obtained from successive assays may be used to show the efficacy of treatment over a period ranging from several days to months.

Results from the methods of the invention may be recorded and/or saved in one or more files or a computer system to facilitate comparison to results from other subjects of known or unknown hypersensitivity and/or clinical condition. The computer system typically includes one or more searchable databases that store and provide information regarding reactions of skin and/or blood tests from the subjects and/or a population of subjects. The computer system can be a stand-alone computer or a conventional network system including a client/server environment and one or more database servers. A number of conventional network systems, including a local area network (LAN) or a wide area network (WAN), are known in the art. Additionally, client/server environments, database servers, and networks are well documented in the technical, trade, and patent literature. For example, the database server can run on an operating system such as UNIX, running a relational database management system, a World Wide Web application, and/or a World Wide Web Server.

As such, in one embodiment, the results from performing the methods of the invention on one or more subjects can be compared to cells that are from the same organ and/or of the same cell type as the subject being tested as a way to confirm the results from the initial test. In another embodiment, the results can be compared to results from corresponding normal cells or cells from a subject that is known to be a low responder in order to identify the subject being tested as a high responder. Thus, the magnitude of the difference in cellular response between a test subject and that of corresponding normal cells or cells from a subject that is known to be a low responder may be indicative of one or more clinical conditions.

As used herein, “corresponding normal cells” refer to a sample of cells obtained from a healthy individual (i.e., a “normal individual”) or an individual known to be a low responder. Such corresponding normal cells can, but need not be, from an individual that is age-matched and/or of the same sex as the individual providing the cells being examined.

Another aspect of the invention provides a kit useful for detecting a clinical condition in a subject. The kit includes one or more anaphylatoxins, such as, for example C3a, C4a, C5a, or analogues thereof. In one embodiment, the anaphylatoxin is as set forth in any one of SEQ ID NOs: 1-92. The kit may further contain a carrier means having at least one container for containing the one or more anaphylatoxins. In another embodiment, the kit may further contain a means for administration of the peptides to a subject, such as, for example, a needle and syringe. In another embodiment, the kit may further contain a means for obtaining the cells of a subject, such as, for example, a needle and syringe, and may further contain a means for contacting the peptides with the cells, such as, for example, a test tube or reaction vessel. Those of ordinary skill in the art will know of other suitable reagents useful for the methods of the invention, inclusion of which is contemplated in the kits of the invention. In one embodiment, the kit also includes a packaging material that can comprise a label which indicates that the anaphylatoxin(s) can be used for detection of hypersensitivity to one or more substances identified above.

Yet another aspect of the invention provides a kit useful for detecting hyper-responsiveness in a subject. The kit includes one or more anaphylatoxins, such as, for example C3a, C5a, or analogues thereof. In one embodiment, the anaphylatoxin is as set forth in any one of SEQ ID NOs: 1-92. The kit may further contain a carrier means having at least one container for containing the one or more anaphylatoxins. In another embodiment, the kit may further contain a means for administration of the peptides to a subject, such as, for example, a needle and syringe. In another embodiment, the kit may further contain a means for obtaining the cells of a subject, such as, for example, a needle and syringe, and may further contain a means for contacting the peptides with the cells, such as, for example, a test tube or reaction vessel. Those of ordinary skill in the art will know of other suitable reagents useful for the methods of the invention, inclusion of which is contemplated in the kits of the invention. In one embodiment, the kit also includes a packaging material that can comprise a label which indicates that the anaphylatoxin(s) can be used for detection of hyper-responsiveness to one or more substances identified above.

Anaphylatoxins of the invention generally cause an immediate, for example, about 5 to 30 minute reaction (e.g., wheal and flare), similar in appearance to the delayed antigen-induced reaction, when they are injected into the skin. Like the antigen response, each individual may react differently (perhaps a 100-fold difference in the cutaneous response in the human population) to the anaphylatoxins.

Previous studies have shown that the cutaneous skin response to synthetic C3a peptides, both in terms of peak response, rate of response and dose dependence, were similar for atopic and non-atopic human subjects. The synthetic C3a peptides used in this study consisted of 10-20 residue fragments based on the C-terminal sequence of human C3a. These data support the conclusion that the cutaneous response to C3a or C3a analogues is independent of the immune-mediated cutaneous response to antigens, otherwise known as an allergic response.

The original discovery that certain C-terminal fragments from the human C3a anaphylatoxin molecule exhibited biological activities identical to the intact natural factor, but with less potency, was reported. The original C-terminal C3a peptides were 8 and 13 residues long and these peptides all retained the exact amino acid sequence of the natural factor. It was hypothesized that the C-terminal pentapeptide sequence of Leu-Gly-Leu-Ala-Arg (C3a 74-77) (SEQ ID NO: 15) was important and possibly essential for activity based on the consensus/conserved sequence in C3a molecules from five different animal species (Table 1A, part II).

Follow-up studies by Caporale et al. (J. Biol. Chem. 255: 10758, 1980) examined the function of length of peptide on activity as well as a number of residue substitutions/modifications of the natural C3a sequence (see Table 1B). Longer peptides were examined for conformational properties, including the 21-residue (C3a 57-77) fragment of human C3a, and it was determined that these longer fragments assumed secondary conformations including a helix (Lu et al. J. Biol. Chem. 259: 7367, 1984). Therefore, it was concluded that the enhanced activity exhibited by the 21-residue fragment was partly due to its ability to fold into a structure that mimics the same region in the natural factor (e.g., the C-terminal helix). (see Table 1A, part I).

TABLE 1A part I

Synthetic Human C3a Peptides Exhibiting Biologic Activity (PNAS,
74, 1826-1830, 1977)

| | | | |
|---|---|---|---|
| | | | Relative Molar |
| Peptide | Structure | Sequence No. | Activity (%) |

| | | | |
|---|---|---|---|
| C3a (1-77) | Natural Factor | — | 100 |
| C3a-(70-77) | H-Ala-Ser-His-Leu-Gly-Leu-Ala- | SEQ ID NO: 1 | 2.0 |
| | Arg-OH | | |
| C3a-(65-77) | H-Arg-Gln-His-Ala-Arg-Ala-Ser- | SEQ ID NO: 2 | 2.5 |
| | His-Leu-Gly-Leu-Ala-Arg-OH | | |
| C3a-(57-77) | H-Cys-Asn-Tyr-Ile-Thr-Glu-Leu- | SEQ ID NO: 3 | >20* |
| | Arg-Arg-Gln-His-Ala-Arg-Ala-Ser- | | |
| | His-Leu-Gly-Leu-Ala-Arg-OH | | |
| E7 | H-Trp-Trp-Gly-Lys-Lys-Tyr-Arg- | SEQ ID NO: 4 | >100** |
| | Ala-Ser-Lys-Leu-Gly-Leu-Ala-Arg- | | |
| | OH | | |

*C3a (57-77) a 21-residue C3a analogue peptide described in J. Biol. Chem. 259: 7367, 1984.
**E7 a 15-residue highly modified C3a analogue peptide described in Biochemistry 30: 3603, 1991.

TABLE 1A, part II

C-terminal sequences and relative activities of
Natural C3a from various animal species

| | | | |
|---|---|---|---|
| Peptide | Sequence | Sequence No. | Potency % |

| | | | |
|---|---|---|---|
| | 70 77 | | |
| C3a (human) | -A-R-A-S-H-L-G-L-A-R | SEQ ID NO: 5 | 100 |
| C3a (guinea pig) | -R-R-E-Q-H-L-G-L-A-R | SEQ ID NO: 6 | 100 |
| C3a (mouse) | -R-R-D-H-V-L-G-L-A-R | SEQ ID NO: 7 | Nd |
| C3a (pig) | -S-R-N-K-P-L-G-L-A-R | SEQ ID NO: 8 | 100 |
| C3a (rat) | -R-R-D-H-V-L-G-L-A-R | SEQ ID NO: 9 | 210 |
| Consensus | R L-G-L-A-R | SEQ ID NO: 10 | — |

C-terminal region of C3a from various animal species is identical indicating that this sequence is essential for C3a-specific function.

TABLE 1B
Synthetic C3a peptides of various lengths and sequences.

| | | | |
|---|---|---|---|
| Peptide Code | Peptide Structure | Sequence No. | Relative Act. % |

| | | | |
|---|---|---|---|
| C3a | Natural human C3a (1-77) | — | 100 |
| 1 | R-Q-H-A-R-A-S-H-L-G-L-A-R | SEQ ID NO: 11 | 5.9 |
| 2 | A-S-H-L-G-L-A-R | SEQ ID NO: 12 | 2.3 |
| 3 | S-H-L-G-L-A-R | SEQ ID NO: 13 | 1.1 |
| 4 | H-L-G-L-A-R | SEQ ID NO: 14 | 0.8 |
| 5 | L-G-L-A-R | SEQ ID NO: 15 | 0.2 |
| 6 | G-L-A-R | SEQ ID NO: 16 | 0.005 |
| 7 | L-A-R | SEQ ID NO: 17 | <0.001 |
| 8 | N-K-P-L-G-L-A-R | SEQ ID NO: 18 | 0.59 |
| 9 | A-A-A-L-G-L-A-R | SEQ ID NO: 19 | 2.0 |
| 10 | A-A-L-G-L-A-R | SEQ ID NO: 20 | 1.2 |
| 11 | A-L-G-L-A-R | SEQ ID NO: 21 | 0.023 |
| 12 | L-G-A-A-R | SEQ ID NO: 22 | <0.003 |
| 13 | formyl-A-S-H-L-G-L-A-R | SEQ ID NO: 23 | 2.0 |
| 14 | formyl-H-L-G-L-A-R | SEQ ID NO: 24 | 0.8 |
| 15 | formyl-L-G-L-A-R | SEQ ID NO: 25 | 0.25 |
| 16 | formyl-A-L-G-L-A-R | SEQ ID NO: 26 | 1.0 |
| 17 | formyl-A-L-G-L-A-K | SEQ ID NO: 27 | <0.005 |

Results show that LGLAR (SEQ ID NO: 15) is the minimal active peptide, is the optimal sequence, and that the C-terminal arginine (Arg, R) is essential for activity. Activity was measured as smooth muscle contraction of the guinea pig ileum. Sequences reported as single letter code for amino acids.

Another follow-up study examined the role of each residue in the pentapeptide (C3a 73-77, Leu-Gly-Leu-Ala-Arg, SEQ ID NO: 15) deemed the minimal active unit of C3a. This study by Unson et al. (Biochemistry, 23:585, 1984) is summarized in Table 1C. A second study using point substitutions in the 21-residue analog peptide (C3a 57-77) was reported by Ember et al. (Biochemistry 30: 3603, 1991) and is shown in part two of Table 1C.

TABLE 1C

Series of substitutions for the C3a pentapeptide
Leu-Gly-Leu-Ala-Arg (C3a 73-77, L-G-L-A-R)

| | | | |
|---|---|---|---|
| | | | Relative molar |
| Peptide | Sequence | Sequence No. | activity (%) |

| | | | |
|---|---|---|---|
| 1 | L-G-L-A-R | SEQ ID NO: 15 | 100 |
| 2 | Y-G-L-A-R | SEQ ID NO: 28 | 160 |
| 3 | F-G-L-A-R | SEQ ID NO: 29 | 140 |
| 4 | I-G-L-A-R | SEQ ID NO: 30 | 100 |
| 5 | V-G-L-A-R | SEQ ID NO: 31 | 90 |
| 6 | M-G-L-A-R | SEQ ID NO: 32 | 50 |
| 7 | A-G-L-A-R | SEQ ID NO: 33 | 20 |
| 8 | L-A-L-A-R | SEQ ID NO: 34 | 270 |
| 9 | L-S-L-A-R | SEQ ID NO: 35 | 55 |
| 10 | L-V-L-A-R | SEQ ID NO: 36 | 10 |
| 11 | L-L-L-A-R | SEQ ID NO: 37 | 10 |
| 12 | L-Q-L-A-R | SEQ ID NO: 38 | <5.5 |
| 13 | L-E-L-A-R | SEQ ID NO: 39 | <2.4 |
| 14 | L-G-I-A-R | SEQ ID NO: 40 | 10 |
| 15 | L-G-M-A-R | SEQ ID NO: 41 | 2.1 |
| 16 | L-G-F-A-R | SEQ ID NO: 42 | <1.5 |
| 17 | L-G-V-A-R | SEQ ID NO: 43 | <1.1 |
| 18 | L-G-A-A-R | SEQ ID NO: 44 | <1.4 |
| 19 | L-G-L-P-R | SEQ ID NO: 45 | 3.5 |
| 20 | L-G-L-Q-R | SEQ ID NO: 46 | 2.8 |
| 21 | L-G-L-S-R | SEQ ID NO: 47 | 0.7 |
| 22 | L-G-L-G-R | SEQ ID NO: 48 | <5.8 |
| 23 | L-G-L-E-R | SEQ ID NO: 49 | <2.6 |

Except for replacing leucine 73 with certain more hydrophobic residues or an alanine for glycine 74, all substitutions resulted in reduced activity. Activity was measured as smooth muscle contraction of guinea pig ileum.

Gerardy-Schahn et al. (1988) introduced the idea of substituting a non-amino acid group to the N-terminal end of the C3a analogue peptides. They reported that a hydrophobic structure such as Fmoc (9-fluorenylmethyloxycarbonyl) or Nap (2-nitro-4-azidophenyl) attached to the N-terminal end of the C3a analogue peptides would enhance their potency. Ember et al. (Biochemistry, 30, 3603, 1991) reported that attaching the hydrophobic amino acid tryptophan (Trp, W) to the N-terminal end of the C3a peptides also markedly enhanced the potency of these peptides (See Tables 1D, 1E, and 1F).

TABLE 1D

Tryptophan and other hydrophobic residue replacements in C3a analogue
peptides.

| | | | Potency |
|---|---|---|---|
| | | | relative to C3a |
| Peptide | Sequence | Sequence No. | (57-77), % |
| | 65 70 77 | | |
| C3a | -R-R-Q-H-A-R-A-S-H-L-G-L-A-R | SEQ ID NO: 50 | — |
| Human | | | |
| B1 | L-G-L-A-R | SEQ ID NO: 15 | 0.2* |
| B2 | R-Y-A-S-K-L-G-L-A-R | SEQ ID NO: 51 | 7.0 |
| B3 | Y-R-A-S-K-L-G-L-A-R | SEQ ID NO: 52 | 55 |
| B4 | Fmoc-Y-R-A-S-K-L-G-L-A-R | SEQ ID NO: 53 | 121 |
| B5 | W-G-G-Y-R-A-S-K-L-G-L-A-R | SEQ ID NO: 54 | 99 |
| B6 | W-W-G-G-Y-R-A-S-K-L-G-L-A-R | SEQ ID NO: 55 | 259 |
| B7 | W-I-G-G-Y-R-A-S-K-L-G-L-A-R | SEQ ID NO: 56 | 118 |
| B8 | I-I-G-G-Y-R-A-S-K-L-G-L-A-R | SEQ ID NO: 57 | 50 |
| B9 | I-I-G-G-Y-R-K-S-A-L-G-L-A-R | SEQ ID NO: 58 | 37 |
| B10 | G-I-G-G-Y-R-K-S-A-L-G-L-A-R | SEQ ID NO: 59 | 41 |
| B11 | I-G-G-G-Y-R-K-S-A-L-G-L-A-R | SEQ ID NO: 60 | 16 |
| B12 | R-R-Y-A-S-K-L-G-L-A-R | SEQ ID NO: 61 | 18 |
| B13 | R-R-R-Y-A-S-K-L-G-L-A-R | SEQ ID NO: 62 | 50 |
| B14 | W-R-R-R-Y-A-S-K-L-G-L-A-R | SEQ ID NO: 63 | 66 |
| B15 | I-R-R-R-Y-A-S-K-L-G-L-A-R | SEQ ID NO: 64 | 99 |
| B16 | W-W-R-R-R-Y-A-S-K-L-G-L-A-R | SEQ ID NO: 66 | 296 |
| B17 | Fmoc-R-R-R-Y-A-S-K-L-G-L-A-R | SEQ ID NO: 66 | 261 |
| B18 | Fmoc-I-R-R--R-Y-A-S-K-L-G-L-A-R | SEQ ID NO: 67 | 50 |
| B19 | Fmoc-W-W-R-R-R-Y-A-S-K-L-G-L-A-R | SEQ ID NO: 68 | 53 |
| B20 | FmocW-R-R-R-Y-A-S-K-L-G-L-A-R | SEQ ID NO: 69 | 70 |

The series of peptides shown in Table 1D shows that analogues can be designed with enhanced potency relative to the natural C3a C-terminal sequence. The enhancement can also be accomplished using natural amino acids rather than non-amino acid groups. The asterisk in Table 1D refers to the fact that the potency of the identified peptide is based on guinea pig ileal assay, whereas all of the other activities were determined using the guinea pig platelet aggregation assay.

TABLE 1E

Potency enhancing effects from adding hydrophobic groups
to the N-terminal end of C3a and C5a analogue peptides.

| | | | |
|---|---|---|---|
| | | | Potency |
| | | | relative to |
| Peptide | Sequence | Sequence No. | C3a (57-77), % |

| | | | |
|---|---|---|---|
| C1 C3a | Y-A-S-K-L-G-L-A-R | SEQ ID NO: 70 | 3.4 |
| analogues | | | |
| C2 | Ahx-Y-A-S-K-L-G-L-A-R | SEQ ID NO: 71 | 13 |
| C3 | Fmoc-Ahx-Y-A-S-K-L-G-L-A-R | SEQ ID NO: 72 | 22 |
| C4 | W-Ahx-Y-A-S-K-L-G-L-A-R | SEQ ID NO: 73 | 22 |
| C5 | Fmoc-Ahx-Y-R-A-S-K-L-G-L-A-R | SEQ ID NO: 74 | 112 |
| C6 | W-W-Ahx-Y-R-A-S-K-L-G-L-A-R | SEQ ID NO: 75 | 603 |
| C7 C5a | Y-S-H-K-G-M-L-L-G-R | SEQ ID NO: 76 | 0.5 |
| analogue | | | |
| C8 | Ahx-Y-S-H-K-G-M-L-L-G-R | SEQ ID NO: 77 | 0.4 |
| C9 | Fmoc-Ahx-Y-S-H-K-G-M-L-L-G-R | SEQ ID NO: 78 | 0.8 |
| C10 | W-Ahx-Y-S-H-K-G-M-L-L-G-R | SEQ ID NO: 79 | 1.2 |

The series of peptides shown in Table 1E indicated that substitution of tryptophan (W) at the N-terminus of analogue C3a peptides was more effective than adding a hydrophobic non-amino acid.

TABLE 1F

An additional series of N-terminal substituted C3a peptides
with multiple amino acid replacements.

| | | | |
|---|---|---|---|
| | | | Potency |
| | | | relative to |
| | | | C3a (57-77) |
| Peptide | Sequence | Sequence No. | % |

| | | | |
|---|---|---|---|
| D1 | Fmoc-A-A-A-R-L-G-L-A-R | SEQ ID NO: 80 | 66 |
| D2 | Fmoc-A-A-R-A-L-G-L-A-R | SEQ ID NO: 81 | 41 |
| D3 | Fmoc-A-R-A-A-L-G-L-A-R | SEQ ID NO: 82 | 37 |
| D4 | Fmoc-R-A-A-A-L-G-L-A-R | SEQ ID NO: 83 | 79 |
| D5 | Fmoc-R-A-A-R-L-G-L-A-R | SEQ ID NO: 84 | 61 |
| D6 | Fmoc-R-R-Y-R-A-S-K-L-G-L-A-R | SEQ ID NO: 85 | 176 |
| D7 | Fmoc-K-K-Y-R-A-S-K-L-G-L-A-R | SEQ ID NO: 86 | 279 |
| D8 | Fmoc-G-G-Y-R-A-S-K-L-G-L-A-R | SEQ ID NO: 87 | 227 |
| D9 | Fmoc-Ahx-Y-R-A-S-K-L-G-L-A-R | SEQ ID NO: 88 | 112 |
| D10 | W-W-G-G-Y-R-A-S-A-L-G-L-A-R | SEQ ID NO: 89 | 181 |
| D11 | W-W-G-G-Y-R-K-S-A-L-G-L-A-R | SEQ ID NO: 90 | 259 |
| D12 | W-W-G-G-Y-R-P-S-A-L-G-L-A-R | SEQ ID NO: 91 | 252 |
| D13 | W-W-G-G-Y-R-a-S-A-L-G-L-A-R | SEQ ID NO: 92 | 118 |

In the series of peptides of Table 1F, “a” denotes D-alanine in D13. This series of peptides provides further information for optimizing the potency of the synthetic C3a analogues using natural amino acids versus non-amino acids.

The following examples are intended to illustrate but not limit the invention.

### Example 1

### Skin Test

The anaphylatoxin agonist peptides, analogue peptides or analogue molecules are synthesized in gram quantities and characterized for purity. Nanogram to microgram quantities of the agonist C3a, C4a or C5a peptide, the C3a, C4a or C5a analogue peptide, or the C3a, C4a or C5a analogue molecule, are dissolved in sterile saline or buffered (phosphate) sterile saline and 25-50 microliters of the solution are injected into the skin. Several concentrations of the agonist peptide, analogue peptide or analogue molecule are injected at different sites to produce a reactivity profile or quantitative indicator of the cutaneous response. The concentrations of agonist peptide, analogue peptide or analogue molecule are selected in a range that will indicate both high and low responders. The relative activity of the agonist peptides or analogue peptides can be estimated using a guinea pig skin test that is visually enhanced by injecting a blue dye prior to challenge.

After injection of the agonist or analogue C3a, C4a or C5a peptide, or the C3a, C4a, or C5a analogue molecule, a visible red skin reaction (wheal and flare) appears within 5-10 min. The diameter or area of the wheal (i.e., raised circular area) or flare (red area) is read (measured in mm) at a fixed time point (i.e., 5-15 min). The cutaneous reaction may cause itching but is generally painless and usually disappears in less than an hour. The high-responders will have a positive (i.e., visible) skin cutaneous reaction to a low test dose of the injected agonist or analogue C3a, C4a or C5a peptide, or analogue C3a, C4a or C5a molecule. The low responders may show no visible response even at the highest test dose of the injected agonist peptide, analogue peptide or analogue molecule. A high-responder having a wheal of 6-10 mm in response to the lowest test dose of agonist or analogue C3a, C4a or C5a peptide or analogue C3a, C4a or C5a molecule would be considered a higher risk individual for severe hypersensitivity (i.e., pseudo-allergic) reactions than a low-responder showing no skin reaction at a low test dose of the effector substance.

Since the anaphylatoxins stimulate the inflammatory cells (including mast cells, basophils, eosinophils, monocytes and neutrophils) to release a spectrum of mediators, a cascade effect can or may be produced. This response can or may be enhanced either by an unusually high cellular response (e.g., “primed cells”) or from an abnormal cell distribution in the skin. When these cellular mediators are released systemically they may themselves produce an abnormal response resulting in an acute or severe response characterized either as a hypersensitivity or anaphylactoid-like reaction.

### Example 2

### Administration of the Skin Test to a Population of Patients Having a History of Hypersensitivity Resulting from a Clinical Treatment

Proof of principle can be obtained using the C3a, C4a, or C5a peptide skin test to evaluate patients who have already experienced a severe (even life threatening) reaction to a clinical treatment such as infused radio-contrast media, infused immunoglobulin therapy, infused protein replacement therapy such as serum albumin, infused recombinant plasma proteins, or general blood substitutes. Comparison skin testing of these hyper-reactive individuals relative to non-responders should reveal the validity of the C3a, C4a or C5a skin test in detecting high responders. If a selected group of individuals, known to have responded with a pseudo-allergic response, exhibit clearly positive (e.g., high responder) C3a, C4a or C5a skin tests, this evidence would support the hypothesis that the skin test does detect non-allergic high responders (e.g., non-immune hypersensitivity or pseudo-allergy). A statistically significant number of test individuals with positive skin test results should provide convincing proof of principle. Any positive correlative result from this type of study would also serve as a proof of principle for the C3a, C4a or C5a peptide skin test.

### Example 3

### Administration of the Skin Test to Additional Individuals

The extent of the variation in a cutaneous response between individuals may be examined in further detail. It has been determined that at least a 10-fold difference in the cutaneous response exists between normal volunteers who were tested using C3a analogue peptides. A more extensive study will include the testing of a larger number of volunteers to determine the magnitude of the difference in skin response in the general population. This study may include testing more than 100 subjects to determine the variations between individuals. It is believed that as much as a 100-fold difference in the C3a, C4a or C5a peptide skin response will be observed in the general population. This is based on our observation that one individual responded dramatically to injection of a much smaller quantity of intact human C5a than was positive in other subjects. This event suggested the present hypothesis that claims a wide variation in responsiveness.

### Example 4

### Blood Test for C3a

The example demonstrates that samples of human blood may be used to detect variable cellular responses to the activating factors C3a or C5a. Targeted blood cell populations for C3a and C3a peptide analogues are basophils and eosinophils because these cells contain C3a receptors and likely also represent (i.e., mimic) the variable (i.e., hyper-responsive) activation state of the skin mast cells.

Five or more individual blood samples are obtained per test group to demonstrate existence of individual variations in responses to C3a/C3a synthetic analogs. All blood incubations are performed at 37° C. The following experiments are performed:

| | | |
|---|---|---|
| 1 | Zero | No additive to the blood samples incubated for 1 to 24 |
| | Control | hours to obtain base-line measurements of background |
| | | cytokine and cell-derived factor profiles including |
| | | eotaxins, IL-5, histamine and many other cell specific |
| | | factors |
| 2 | Plummer's SCPN | 1 to 24 hour incubation profile of blood-derived |
| | Inhibitor Control | cytokines and other cellular factors measured in |
| | | Plummer's SCPN inhibitor control samples. Various |
| | | levels of background cell-derived factors may be |
| | | released by factors such as C3a or C5a that may be |
| | | generated during incubation of the blood. This |
| | | experiment is designed to measure background levels |
| | | of cell-derived factors released when the control |
| | | enzyme SCPN is inhibited. Add Plummer's Inhibitor |
| | | to 1 microgram/ml of final blood volume (3 |
| | | micromolar or 3,000 × ID₅₀) and begin incubation. * |
| 3 | EACA SCPN | Add EACA to 1 mg/ml of final blood volume (3 |
| | Inhibitor Control | millimolar or 3,000 × ID₅₀) then begin incubation. |
| | | Obtain 1 to 24 hour profiles of cytokines and other cell- |
| | | derived factors to determine EACA inhibitor control |
| | | levels. * |
| 4 | Plummer's SCPN | Add 1 microgram Inhibitor/ml of final blood volume |
| | Inhibitor + C3a | first, then 1-5 minutes later add 10 micrograms of |
| | Positive Control | C3a/ml of final blood volume and begin incubation. |
| | | Obtain 1 to 24 hour profiles of cytokines and other cell- |
| | | derived factors to determine C3a release levels. ** |
| 5 | EACA SCPN | Add 1 milligram Inhibitor/ml of final blood volume |
| | Inhibitor + C3a | first, then 1-5 minutes later add 10 micrograms of |
| | Positive Control | C3a/ml of final blood volume and begin incubation. |
| | | Obtain 1 to 24 hour profiles of cytokines and other cell- |
| | | derived factors to determine C3a release levels. ** |
| 6 | Plummer's SCPN | Add 1 microgram Inhibitor/ml of final blood volume |
| | Inhibitor + C3a 8R | first, then 1-5 minutes later add 10 micrograms of C3a |
| | | 8R peptide/ml of final blood volume and begin |
| | | incubation. Obtain 1 to 24 hour profiles of cytokines |
| | | and other cell-derived factors to determine C3a 8R |
| | | (i.e., C3a C-terminal octapeptide analog) release levels. |
| | | *** |
| 7 | EACA SCPN | Add 1 milligram Inhibitor/ml of final blood volume |
| | Inhibitor + C3a 8R | first, then 1-5 minutes later add 10 micrograms of C3a |
| | | 8R peptide/ml of final blood volume and begin |
| | | incubation. Obtain 1 to 24 hour profiles of cytokines |
| | | and other cell-derived factors to determine C3a 8R |
| | | release levels. *** |
| 8 | C3a Control | Add 10 micrograms of C3a/ml final blood volume and |
| | | begin incubation. Obtain 1 to 24 hour profiles of |
| | | cytokines and other cell-derived factors to determine |
| | | C3a release levels when SCPN is not inhibited. +++ |

Experiment 1 will provide the background levels of all cell-derived factors measured (i.e., the noise level). Note that time points for measuring these factors may be selected between 1 and 24 hours and the cell-derived factors measured may be selected from all factors released by blood cells. * Experiments 2 and 3 are designed to determine the effects of inhibiting serum carboxypeptidase N(SCPN) and the potential effects of each inhibitor on cellular release. The suggested dose levels of these inhibitors are based on known ID₅₀ values, assuming that a 3000-fold level will control the SCPN long enough to allow the C3a/C3a peptides to act on the blood cells. Plummer's inhibitor is DL-2-mercaptomethyl-3-guanidinoethylthiopropanoic acid, and EACA is 6-aminohexanoic acid. ** Experiments 4 and 5 are C3a positive controls. 10 μg C3a/ml is about 15% of full (i.e., 60 μg C3a/ml) complement activation in blood, which is considered to be a high level of C3a for cellular activation. *** Experiments 6 and 7 are designed to test the activity of the C3a synthetic analog peptides. +++Experiment 8 is designed to determine if C3a has an effect in blood when SCPN is not inhibited. This may be informative if C3a is able to stimulate cells at some level before it is inactivated by the SCPN.

In order to normalize these cellular responses to actual cell numbers, blood cell counts should be obtained for the individual blood donors.

### Example 5

### Blood Test for C5a

This example is designed to demonstrate/determine blood cell responses and variations when stimulated/activated by C5a or C5a synthetic analogs. Experiments 1-8 from Example 4 will be repeated using C5a or a C5a synthetic analog instead of C3a or C3a 8R, respectively. The design is identical and the cell-derived factor profiles obtained is similar except that leukocyte- and monocyte-specific cytokines are measured in addition to basophil eosinophil factors, because the leukocytes and monocytes contain C5a receptors and respond to C5a.

### Example 6

### Blood Testing of a Population

This example is designed to test large numbers (e.g. >100) of individual blood samples for stimulation/activation by C3a (or C3a analogs) and C5a (or C5a analogs) to determine the extent of the variation among individuals. Experiments 1-8 from Example 4 are repeated essentially the same as in Example 4 for C3a and Example 5 for C5a to obtain data that will indicate the extent of the variations in individual blood samples stimulated/activated by C3a or C5a.

### Example 7

### Blood Testing of Specific Groups

This example is designed to test blood samples from specific groups of individuals with identified or known clinical conditions. The goal is to determine if these groups have higher or lower levels of blood cell factor release by C3a or C5a stimulation/activation than the general population or other select groups. Experiments 1-8 from Example 4 are repeated essentially the same as in Example 4 for C3a and Example 5 for C5a to obtain data that can be normalized by control or background release levels. These data will help confirm and/or identify specific clinical conditions or states that the blood assays will or can detect.

### REFERENCES

Szebeni, J. (2001) Complement activation-related pseudoallergy caused by liposomes, micellar carriers of intravenous drugs, and radiocontrast agents, Crit. Rev. Ther. Drug Carrier Syst. 18, 567-606.
Szebeni, J., Alving, C. R., Savay, S., Barenholz, Y., Priev, A. Damino, D. and Talmon, Y (2001) Formation of complement-activating particles in aqueous solutions of Taxol: possible role in hypersensitivity reactions. Int. Immunopharmacol. 1, 721-735.
Hugli, T. E. Human anaphylatoxin (C3a) from the third component of complement: Primary structure. J. Biol. Chem. 250: 8293-8301, 1975.
Hugli, T. E. and Erickson, B. W. Synthetic peptides with the biological activities and specificity of human C3a anaphylatoxin. Proc. Natl. Acad. Sci. USA. 74: 1826-1830, 1977.
Caporale, L. H., Erickson, B. W. and Hugli, T. E. Synthetic oligopeptides from human C3a anaphylatoxin that mediate the inflammatory response. Proceedings of the Fifth American Peptide Symposium, M. Goodman and J. Meienhofer, Eds., pp 225-227, (Halstead Press, New York) 1977.
Hugli, T. E. Chemical aspects of the serum anaphylatoxins. In: Contemporary Topics in Molecular Immunology, vol. 7, R. A. Reisfeld and F. Inman, Eds., pp. 181-214 (Plenum Press, New york) 1978.
Unson, C. G., Erickson, B. W. and Hugli, T. E. Role of leucine residues in the active site of C3a anaphylatoxin. In: Peptides: Structure and Biological Function, E. Gross and J. Meienhofer, Eds., pp. 459-462 (Pierce Chemical Co., Rockford, Ill.) 1979.
Caporale, L. H., Tippett, P. S., Erickson, B. W. and Hugli, T. E. The active site of C3a anaphylatoxin. J. Biol. Chem. 255: 10758-10763, 1980.
Lu, Z-Xian, Fok, K. F., Erickson, B. W. and Hugli, T. E. Conformational analysis of COOH-terminal fragments of human C3a: Evidence of ordered conformation in an active monocosapeptide. J. Biol. Chem. 259: 7367-7370, 1984.
Unson, C. G., Erickson, B. W. and Hugli, T. E. Active site of C3a anaphylatoxin: contribution of the lipophilic and orienting residues. Biochemistry 23: 585-589, 1984.
Hoeprich, P. D. and Hugli, T. E. Helical conformation of carboxyl terminus of human C3a is required for full activity. Biochemistry 25: 1945-1950, 1986.
Hugli, T. E. Structure and function of C3a anaphylatoxin. In: Current Topics in Microbiology and Immunology, Vol. 153, Components of Complement (John D. Lambris and Hans J. Muller-Eberhard, Eds (Springer-Verlag, Berlin-Heidelberg) pp. 181-208, 1989.
Ember, J. A., Johansen, M. L. and Hugli, T. E. Designing synthetic supra-agonists of C3a anaphylatoxin. Biochemistry 30: 3603-3612, 1991.
Ambrosius, D., Casaretto, M., Gerardy-Schahn, R., Sanders, D., Brandenburg, D., and Kahn, H. Peptide analogues of the anaphylatoxin C3a: synthesis and properties. Biol. Chem. Hoppe-Seyler, 370: 217-227, 1989.
Gerardy-Schahn R., Ambrosius D., Casaretto M., Grotzinger J., Sanders, D., Woller, A., Brandenburg, D., and Bitter-Suermann, D. Design and biological activity of a new generation of synthetic C3a analogues by combination of peptidic and non-peptidic elements. Biochem. J. 255: 209-216, 1988.
Gerardy-Schahn, R., Ambrosius, D., Sanders, D., Casaretto, M, Mittler, C., Karwath, G., Goren, S., and Bitter-Suermann, D. Eur. J. Immunol. 19:1095-1102, 1989.
Corbin, N. C. and Hugli, T. E. The primary structure of porcine C3a anaphylatoxin. J. Immunol. 117: 990-995, 1976.
Jacobs, J. W., Rubin, J. S., Hugli, T. E., Bogardt, R. A., Mariz, I. K., Daniels, J. S., Daughaday, W. H., and Bradshaw, R. A. Purification, characterization and amino acid sequence of rat anaphylatoxin (C3a). Biochemistry 17: 5031-5038, 1978.
Glovsky, M. M., Hugli, T. E., Hartman, C. T. and Ghekiere, L. Possible role of C3a in human disease. In: Clinical aspects of the complement system, W. Opferkuch and K. Rother, Eds., pp 138-144, (Pieme Verlag Publishers, Stuttgart, West Germany, 1978.
Glovsky, M. M., Hugli, T. E., Ishizaka, T., Lichenstein, L. M. and Erickson, B. W. Anaphylatoxin-induced histamine release with human leukocytes: Studies of C3a leukocyte binding and histamine release. J. Clin. Invest. 64: 804-811, 1979.
Gorski, J. P., Hugli, T. E. and Muller-Eberhard, H. J. Characterization of human C4a anaphylatoxin. J. Biol. Chem. 256: 2707-2711, 1981.
Moon, K. E., Gorski, J. P. and Hugli, T. E. Complete primary structure of human C4a anaphylatoxin. J. Biol. Chem. 256: 8685-8692, 1981.
Hugli, T. E., Kawahara, M. S., Unson, C. G., Molinor, R. L. and Erickson, B. W. The active site of human C4a anaphylatoxin. Molecular Immunology 20: 637-645, 1983.
Huey, R., Erickson, B. W., Bloor, C. M. and Hugli, T. E. Contraction of guinea pig lung by synthetic oligopeptides related to human C3a. Immunopharmacology 8: 37-45, 1984.
Morgan, E. L., Weigle, W. O., Erickson, B. W., Fok, K-F., and Hugli, T. E. Suppression of humoral immune responses by synthetic C3a peptides. J. Immunol. 131: 2258-2261, 1983.
Marceau, F. M. and Hugli, T. E. Effect of C3a and C5a anaphylatoxins on guinea pig isolated blood vessels. J. Pharma. and Exp. Therapeutics 230: 749-754, 1984.
Bjork, J., Hugli, T. E. and Smedegard, G. Microvascular effects of anaphylatoxins C3a and C5a. J. Immunol. 134: 1115-1119, 1985.
Marceau, F., Lundberg, C. and Hugli, T. E. Effects of the anaphylatoxins on circulation (Short Review). Immunopharmacology 14: 67-84, 1987.
Cui, L-X., Ferreri, K. and Hugli, T. E. Structural characterization of the C4a anaplylatoxin from rat. Molecular Immunology 25: 663-671, 1988.
Fukuoka, Y. and Hugli, T. E. Demonstration of specific C3a receptors on guinea pig platelets. J. Immunol. 140: 3496-3501, 1988.
Fukuoka, Y., Nielsen, L. P. and Hugli, T. E. Characterization of receptors to the anaphylatoxins on isolated cells. Dermatologia 179 (suppl 1): 25-40, 1989.
Fukuoka, Y. and Hugli, T. E. Anaphylatoxin binding and degradation by rat peritoneal mast cells. J. Immunol. 145: 1851-1858, 1990.
Ember, J. A., Johansen, N. L. and Hugli, T. E. A new approach to designing active analogues of proteins. Biochem. Soc. Trans. 18: 1143-1145, 1990.
Kajita, T. and Hugli, T. E. Evidence for in vivo degradation of C3a anaphylatoxin by mast cell chymase: I. Non-specific activation of rat peritoneal mast cells by C3a des Arg. Amer. J. Path. 138: 1359-1369, 1991.
Mousli, M., Hugli, T. E., Landry, Y. and Bronner, C. A mechanism for anaphylatoxin C3a stimulation of mast cells. J. Immunol. 148: 2456-2461, 1992.
Ember, J. A., Sanderson, D. G., Taylor, S., Kawahara, M. and Hugli, T. E. Biologic activity of synthetic analogs of C5a anaphylatoxin. J. Immunol. 148: 3165-3173, 1992.
Morgan, E. L, Sanderson, S. D., Scholz, W., Noonan, D. J., Weigle, W. O. and Hugli, T. E. Identification and characterization of the effector region within human C5a responsible for stimulation of interleukin-6 synthesis. J. Immunol. 148: 3937-3942, 1992.
Cui, L-X., Carney, D. F. and Hugli, T. E. Primary structure and functional characterization of rat C5a: An anaphylatoxin with unusually high potency. Protein Sci. 3: 1169-1177, 1994.

Although the invention has been described with reference to the above examples, it will be understood that modifications and variations are encompassed within the spirit and scope of the invention. Accordingly, the invention is limited only by the following claims.

What is claimed is:

1. A method of detecting a clinical condition in a subject comprising:
(a) contacting a sample of cells from the subject with an anaphylatoxin; and
(b) detecting a cellular reaction,
wherein the reaction is indicative of a clinical condition in a subject.

2. The method of claim 1, wherein the method is performed ex vivo.

3. The method of claim 1, wherein the cells are blood cells.

4. The method of claim 1, wherein the cellular reaction is expression of one or more cell factors selected from the group consisting of a cytokine, a metabolic factor and a cell marker.

5. The method of claim 1, wherein the anaphylatoxin is C3a, C4a, C5a, or analogues thereof.

6. The method of claim 5, wherein the anaphylatoxin is a peptide.

7. The method of claim 6, wherein the peptide is any one of SEQ ID NOs: 1-92.

8. The method of claim 1, wherein the anaphylatoxin is a small molecule.

9. The method of claim 1, wherein the detecting occurs within 1-24 hours of contact.

10. The method of claim 1, further comprising contacting the cells with an inhibitor of serum carboxypeptidase N (SCPN).

11. The method of claim 10, wherein the inhibitor is DL-2-mercaptomethyl-3-guanidinoethylthiopropanoic acid.

12. The method of claim 10, wherein the inhibitor is 6-aminohexanoic acid.

13. The method of claim 1, wherein the cellular reaction is indicative of systemic hyper-responsiveness.

14. The method of claim 1, wherein the cellular reaction is indicative of pseudo-allergy.

15. The method of claim 1, wherein the cellular reaction is indicative of one or more clinical conditions selected from the group consisting of asthma, inflammation, chronic or acute infection, bacterial infection, viral infection, parasite infection, adverse drug reaction, organ rejection, vasculitis, mastocytosis, eosinophilia, basophilia, leukemia, and C3a or C5a receptor defects.

16. The method of claim 1, wherein the cellular reaction is indicative of abnormal reactivity towards infused recombinant proteins.

17. The method of claim 16, wherein the recombinant protein is an immunoglobulin or an antibody.

18. The method of claim 1, further comprising administering the anaphylatoxin to the subject and detecting a non-allergic reaction in the subject.

19. The method of claim 1, further comprising comparing the detected cellular reaction to a cellular reaction from cells of a corresponding normal subject or from a subject of known clinical condition, wherein a difference in the cellular reaction is indicative of the clinical condition.

20. A kit for detecting a clinical condition in a subject comprising at least one anaphylatoxin selected from C3a, C5a, or analogues thereof, and a means for obtaining cells from the subject.

21. The kit of claim 20, wherein the anaphylatoxin is SEQ ID NO: 1-92.

22. The kit of claim 20, further comprising a means for contacting the anaphylatoxin with the cells.


SEQUENCE LISTING (92 sequences)

SEQ ID NO: 1 - protein, 8 aa, Artificial sequence
  Synthetic construct
ASHLGLAR

SEQ ID NO: 2 - protein, 13 aa, Artificial sequence
  Synthetic construct
RQHARASHLGLAR

SEQ ID NO: 3 - protein, 21 aa, Artificial sequence
  Synthetic construct
CNYITELRRQHARASHLGLAR

SEQ ID NO: 4 - protein, 15 aa, Artificial sequence
  Synthetic construct
WWGKKYRASKLGLAR

SEQ ID NO: 5 - protein, 10 aa, Homo sapiens
ARASHLGLAR

SEQ ID NO: 6 - protein, 10 aa, Guinea pig
RREQHLGLAR

SEQ ID NO: 7 - protein, 10 aa, Mouse
RRDHVLGLAR

SEQ ID NO: 8 - protein, 10 aa, Pig
SRNKPLGLAR

SEQ ID NO: 9 - protein, 10 aa, Rat
RRDHVLGLAR

SEQ ID NO: 10 - protein, 6 aa, Artificial sequence
  Consensus sequence
RLGLAR

SEQ ID NO: 11 - protein, 13 aa, Artificial sequence
  Synthetic construct
RQHARASHLGLAR

SEQ ID NO: 12 - protein, 8 aa, Artificial sequence
  Synthetic construct
ASHLGLAR

SEQ ID NO: 13 - protein, 7 aa, Artificial sequence
  Synthetic construct
SHLGLAR

SEQ ID NO: 14 - protein, 6 aa, Artificial sequence
  Synthetic construct
HLGLAR

SEQ ID NO: 15 - protein, 5 aa, Artificial sequence
  Synthetic construct
LGLAR

SEQ ID NO: 16 - protein, 4 aa, Artificial sequence
  Synthetic construct
GLAR

SEQ ID NO: 17 - protein, 3 aa, Artificial sequence
  Synthetic construct
LAR

SEQ ID NO: 18 - protein, 8 aa, Artificial sequence
  Synthetic construct
NKPLGLAR

SEQ ID NO: 19 - protein, 8 aa, Artificial sequence
  Synthetic construct
AAALGLAR

SEQ ID NO: 20 - protein, 7 aa, Artificial sequence
  Synthetic construct
AALGLAR

SEQ ID NO: 21 - protein, 6 aa, Artificial sequence
  Synthetic construct
ALGLAR

SEQ ID NO: 22 - protein, 5 aa, Artificial sequence
  Synthetic construct
LGAAR

SEQ ID NO: 23 - protein, 8 aa, Artificial sequence
  Synthetic construct
ASHLGLAR

SEQ ID NO: 24 - protein, 6 aa, Artificial sequence
  Synthetic construct
HLGLAR

SEQ ID NO: 25 - protein, 5 aa, Artificial sequence
  Synthetic construct
LGLAR

SEQ ID NO: 26 - protein, 6 aa, Artificial sequence
  Synthetic construct
ALGLAR

SEQ ID NO: 27 - protein, 6 aa, Artificial sequence
  Synthetic construct
ALGLAK

SEQ ID NO: 28 - protein, 5 aa, Artificial sequence
  Synthetic construct
YGLAR

SEQ ID NO: 29 - protein, 5 aa, Artificial sequence
  Synthetic construct
FGLAR

SEQ ID NO: 30 - protein, 5 aa, Artificial sequence
  Synthetic construct
IGLAR

SEQ ID NO: 31 - protein, 5 aa, Artificial sequence
  Synthetic construct
VGLAR

SEQ ID NO: 32 - protein, 5 aa, Artificial sequence
  Synthetic construct
MGLAR

SEQ ID NO: 33 - protein, 5 aa, Artificial sequence
  Synthetic construct
AGLAR

SEQ ID NO: 34 - protein, 5 aa, Artificial sequence
  Synthetic construct
LALAR

SEQ ID NO: 35 - protein, 5 aa, Artificial sequence
  Synthetic construct
LSLAR

SEQ ID NO: 36 - protein, 5 aa, Artificial sequence
  Synthetic construct
LVLAR

SEQ ID NO: 37 - protein, 5 aa, Artificial sequence
  Synthetic construct
LLLAR

SEQ ID NO: 38 - protein, 5 aa, Artificial sequence
  Synthetic construct
LQLAR

SEQ ID NO: 39 - protein, 5 aa, Artificial sequence
  Synthetic construct
LELAR

SEQ ID NO: 40 - protein, 5 aa, Artificial sequence
  Synthetic construct
LGIAR

SEQ ID NO: 41 - protein, 5 aa, Artificial sequence
  Synthetic construct
LGMAR

SEQ ID NO: 42 - protein, 5 aa, Artificial sequence
  Synthetic construct
LGFAR

SEQ ID NO: 43 - protein, 5 aa, Artificial sequence
  Synthetic construct
LGVAR

SEQ ID NO: 44 - protein, 5 aa, Artificial sequence
  Synthetic construct
LGAAR

SEQ ID NO: 45 - protein, 5 aa, Artificial sequence
  Synthetic construct
LGLPR

SEQ ID NO: 46 - protein, 5 aa, Artificial sequence
  Synthetic construct
LGLQR

SEQ ID NO: 47 - protein, 5 aa, Artificial sequence
  Synthetic construct
LGLSR

SEQ ID NO: 48 - protein, 5 aa, Artificial sequence
  Synthetic construct
LGLGR

SEQ ID NO: 49 - protein, 5 aa, Artificial sequence
  Synthetic construct
LGLER

SEQ ID NO: 50 - protein, 14 aa, Homo sapiens
RRQHARASHLGLAR

SEQ ID NO: 51 - protein, 10 aa, Artificial sequence
  Synthetic construct
RYASKLGLAR

SEQ ID NO: 52 - protein, 10 aa, Artificial sequence
  Synthetic construct
YRASKLGLAR

SEQ ID NO: 53 - protein, 10 aa, Artificial sequence
  Synthetic construct
YRASKLGLAR

SEQ ID NO: 54 - protein, 13 aa, Artificial sequence
  Synthetic construct
WGGYRASKLGLAR

SEQ ID NO: 55 - protein, 14 aa, Artificial sequence
  Synthetic construct
WWGGYRASKLGLAR

SEQ ID NO: 56 - protein, 14 aa, Artificial sequence
  Synthetic construct
WIGGYRASKLGLAR

SEQ ID NO: 57 - protein, 14 aa, Artificial sequence
  Synthetic construct
IIGGYRASKLGLAR

SEQ ID NO: 58 - protein, 14 aa, Artificial sequence
  Synthetic construct
IIGGYRKSALGLAR

SEQ ID NO: 59 - protein, 14 aa, Artificial sequence
  Synthetic construct
GIGGYRKSALGLAR

SEQ ID NO: 60 - protein, 14 aa, Artificial sequence
  Synthetic construct
IGGGYRKSALGLAR

SEQ ID NO: 61 - protein, 11 aa, Artificial sequence
  Synthetic construct
RRYASKLGLAR

SEQ ID NO: 62 - protein, 12 aa, Artificial sequence
  Synthetic construct
RRRYASKLGLAR

SEQ ID NO: 63 - protein, 13 aa, Artificial Sequence
  Synthetic construct
WRRRYASKLGLAR

SEQ ID NO: 64 - protein, 13 aa, Artificial sequence
  Synthetic construct
IRRRYASKLGLAR

SEQ ID NO: 65 - protein, 14 aa, Artificial sequence
  Synthetic construct
WWRRRYASKLGLAR

SEQ ID NO: 66 - protein, 12 aa, Artificial sequence
  Synthetic construct
RRRYASKLGLAR

SEQ ID NO: 67 - protein, 13 aa, Artificial sequence
  Synthetic construct
IRRRYASKLGLAR

SEQ ID NO: 68 - protein, 14 aa, Artificial sequence
  Synthetic construct
WWRRRYASKLGLAR

SEQ ID NO: 69 - protein, 13 aa, Artificial sequence
  Synthetic construct
WRRRYASKLGLAR

SEQ ID NO: 70 - protein, 9 aa, Artificial sequence
  Synthetic construct
YASKLGLAR

SEQ ID NO: 71 - protein, 9 aa, Artificial sequence
  Synthetic construct
YASKLGLAR

SEQ ID NO: 72 - protein, 9 aa, Artificial sequence
  Synthetic construct
YASKLGLAR

SEQ ID NO: 73 - protein, 11 aa, Artificial sequence
  Synthetic construct
WXYASKLGLAR

SEQ ID NO: 74 - protein, 10 aa, Artificial sequence
  Synthetic construct
YRASKLGLAR

SEQ ID NO: 75 - protein, 13 aa, Artificial sequence
  Synthetic construct
WWXYRASKLGLAR

SEQ ID NO: 76 - protein, 10 aa, Artificial sequence
  Synthetic construct
YSHKGMLLGR

SEQ ID NO: 77 - protein, 10 aa, Artificial sequence
  Synthetic construct
YSHKGMLLGR

SEQ ID NO: 78 - protein, 10 aa, Artificial sequence
  Synthetic construct
YSHKGMLLGR

SEQ ID NO: 79 - protein, 12 aa, Artificial sequence
  Synthetic construct
WXYSHKGMLLGR

SEQ ID NO: 80 - protein, 9 aa, Artificial sequence
  Synthetic construct
AAARLGLAR

SEQ ID NO: 81 - protein, 9 aa, Artificial sequence
  Synthetic construct
AARALGLAR

SEQ ID NO: 82 - protein, 9 aa, Artificial sequence
  Synthetic construct
ARAALGLAR

SEQ ID NO: 83 - protein, 9 aa, Artificial sequence
  Synthetic construct
RAAALGLAR

SEQ ID NO: 84 - protein, 9 aa, Artificial sequence
  Synthetic construct
RAARLGLAR

SEQ ID NO: 85 - protein, 12 aa, Artificial sequence
  Synthetic construct
RRYRASKLGLAR

SEQ ID NO: 86 - protein, 12 aa, Artificial sequence
  Synthetic construct
KKYRASKLGLAR

SEQ ID NO: 87 - protein, 12 aa, Artificial sequence
  Synthetic construct
GGYRASKLGLAR

SEQ ID NO: 88 - protein, 10 aa, Artificial sequence
  Synthetic construct
YRASKLGLAR

SEQ ID NO: 89 - protein, 14 aa, Artificial sequence
  Synthetic construct
WWGGYRASALGLAR

SEQ ID NO: 90 - protein, 14 aa, Artificial sequence
  Synthetic construct
WWGGYRKSALGLAR

SEQ ID NO: 91 - protein, 14 aa, Artificial sequence
  Synthetic construct
WWGGYRPSALGLAR

SEQ ID NO: 92 - protein, 14 aa, Artificial sequence
  Synthetic construct
WWGGYRASALGLAR